(19) United States
(12) Patent Application Publication
(10) Pub. No.: US 2026/0265777 A1
(43) Pub. Date: Sep. 10, 2026

(54) EXPRESSION CONSTRUCT

(71) Applicant: Lentitek Ltd, Edinburgh (GB)

(72) Inventors: Adam Inche, Edinburgh (GB); Leif Anderson, Edinburgh (GB)

(21) Appl. No.: 19/167,674

(22) PCT Filed: Mar. 22, 2024
(86) PCT No.: PCT/GB2024/050776

§ 371 Date: Sep. 22, 2025

(30) Foreign Application Priority Data
Mar. 22, 2023 (GB) 2304141.1

(51) Int. Cl. C12N 15/86; C07K 14/005; C07K 14/435

(52) U.S. Cl. CPC C12N 15/86; C07K 14/005; C07K 14/43595; C12N 2740/13022; C12N 2740/15043; C12N 2740/15052; C12N 2760/18422; C12N 2760/20122; C12N 2760/20222

(57) ABSTRACT

The disclosure relates to constructs, especially lentivral constructs for use in an expression system for cell and/or gene therapy applications. The constructs include a viral genome, the expression of which is controlled by an RNA polymerase I promoter.

### FIELD

The present disclosure relates to constructs for use in an expression system for cell and/or gene therapy applications.

### REFERENCE TO AN ELECTRONIC SEQUENCE LISTING

The contents of the electronic sequence listing (PE962042WO_-_ST26_Sequence_Listing.xml; Size: 10,024 bytes; and Date of Creation: Sep. 9, 2025) is herein incorporated by reference in its entirety.

### BACKGROUND

There are a number of viral vectors for gene therapy, which have been successfully employed within patients to treat diseases. These viral vectors are typically based on adenovirus, adeno associated virus (AAV), or retrovirus [1]. The most common retrovirus used for gene therapies is the lentivirus, as they can transduce both dividing and non-dividing cells. Vector platforms based on Equine Infectious Anaemia Virus (EIAV) and Human Immunodeficiency Virus (HIV) have been used, with HIV being the most common lentivirus system in clinical and commercial use today [2]. Indeed, all of the Chimeric Antigen Receptor T-cell (CAR-T) therapies approved globally in 2022 use an HIV-based lentiviral vector within the manufacturing process to reprogram the T-cell.

Lentiviral vectors, in line with wild type lentiviruses, are single-stranded positive sense RNA viruses where the virion is enveloped [2]. Lentivirus can transduce dividing and non-dividing cells, leading to sustained expression of the gene within either types of cell [1]. AAV can also infect non-dividing cells, however as a much smaller virus, only smaller portions of genetic material can be transduced, discounting this system for many applications like CAR-T [2]. Lentiviral manufacturing systems typically use a triple transfection system. For safety, the plasmids containing the packaging and envelope components are separate from the viral vector genome. Where it is possible for all of the components to produce the lentivirus to be contained within three, sometimes four, plasmids; manufacturers will often include specific elements within a manufacturing cell line, termed a packaging cell line [2].

Commonly used approaches in the art for the production of the retroviral vectors generally involve the use of plasmids containing an RNA polymerase II promoter driving the production of both the viral protein components as well as the viral genome. The CMV promoter is a common choice as it has very high expression levels for proteins in mammalian cells [3]. However, RNA polymerase II promoter typically terminates transcripts with a poly(A) tail, which directs these transcripts for translation into proteins [4]. In the context of gene therapy applications, translation of the transcripts into proteins in the manufacturing cell line is likely to be disadvantageous as the breakthrough expression of these proteins may result in moderate to even severe toxicity [5], or other breakthrough expression consequences [6]. For instance, it has previously been demonstrated in the art that CD19 CARs can be presented on the surface of lentiviral particles due to expression of the transgene during manufacture, leading to off-target transduction of B cells and relapse of B cell lymphoma [6].

Alternatively, or in addition, an RNA polymerase I promoter may be used to produce the viral protein components and the viral vector genome. For example, EP1317559 discloses a DNA transfection system for the generation of infectious negative strand RNA virus for use in developing recombinant influenza vaccines. The plasmids described therein comprise both a polymerase I promoter and a polymerase II promoter, a polyadenylation signal, and viral cDNA(s) transcribed from the two promoters for the generation of infectious negative strand RNA viruses, such as influenza viruses. However, EP1317559 and other viral vector systems disclosed in the art deploying RNA polymerase I promoters typically produce replicating infectious recombinant viruses for use as vaccines. Thus, there is still an unmet need for safe expression systems and methods for efficient production of vectors for use in such expression systems that are suitable for cell and/or gene therapy applications. In particular, there is a need for a novel system for manufacturing engineered non-replicating retroviruses, such as lentiviruses, with enhanced efficiency, reduced toxicity or other breakthrough expression consequences and/or improved safety.

### SUMMARY

The present disclosure is based in part on the finding that RNA polymerase I (Pol I) may be used to drive the efficient and improved generation of a retrovirus, especially lentivirus genome in a cell.

In one aspect there is provided a lentiviral genome construct comprising a lentiviral genome and a RNA polymerase I promoter, wherein expression of the lentiviral genome is under control of the RNA polymerase I promoter.

Without wishing to be bound by theory, transcripts generated using RNA polymerase I (Pol I) are not modified by the addition of a poly-A tail, thereby ensuring that (in the cell) the transcript is available for its intended purpose and not inappropriately directed to the cell protein production machinery (e.g. the Golgi apparatus). Generating transcripts using Pol I may also reduce the potential for breakthrough expression of the transcript (or a portion or portions thereof) in the cell and/or inappropriate RNA splicing (see for example Cooper et al., Nucleic Acids Research, Volume 43, Issue 1, 9 Jan. 2015, Pages 682-690.

In one teaching, the various methods, uses and systems described herein may be exploited in the manufacture of viral vectors, especially lentiviral vectors. In such applications, at least the viral genome may be placed under the control of Pol I; this ensures efficient generation of the viral genome transcript in a manufacturing cell line. Moreover, the use of Pol I to generate a viral (for example lentiviral) vector genome, ensures that the generated transcript is available for packaging and is not inappropriately directed to the cell's protein production machinery. Taken together, these advantages deliver an improved and more efficient method for the production of viral vectors, including, for example, lentiviral vectors.

In view of the above, in one aspect, the disclosure provides an improved method for the generation of retroviral genome, especially lentiviral genome transcripts in a cell, said method comprising
(i) operatively linking a retroviral genome sequence for transcription in a cell to a RNA Polymerase I promoter (Pol I); and/or
(ii) placing a retroviral genome sequence for transcription in a cell under the control of a RNA Polymerase I promoter (Pol I).

The method of this disclosure is improved because:
(a) the transcript yield is improved;
(b) the amount of transcript available for its intended purpose (packaging into a viral vector) is increased;
(c) the risk of breakthrough expression may be reduced; and/or
(d) where the transcript is necessary for the manufacture of a viral (e.g. lentiviral) vector genome, the manufacture is made more efficient and yield of the viral vector is improved.

As previously explained and without wishing to be bound by theory, these improvements stem from the fact that transcripts generated using Pol I promoters are not modified to include a poly-A tail and this avoids the transcript being inappropriately directed to a cell's protein making machinery and/or being inappropriately spliced. This increases the amount of transcript available for its intended purpose. Where the transcript is a viral vector genome (for example a lentiviral vector genome) that purpose is for the genome transcript to be packaged into a viral vector (with glycoprotein and viral protein components) for use.

In the context of this disclosure, the term “transcript” may embrace any RNA copy of a piece of DNA. The DNA may encode a protein, for example a therapeutic protein or it may be non-coding in that it does not encode a protein or peptide.

Together with direct viral yield improvement, a construct for producing retroviruses comprising an RNA polymerase I (Pol I) promoter in combination with long terminal repeat sequences could provide an improved system with enhanced efficiency, reduced host cell toxicity or other issues associated with breakthrough expression of the payload or transgene. Importantly, the constructs of the present disclosure are suitable for the production of non-replicating recombinant retroviruses for use in cell and/or gene therapy applications. This disclosure provides a lentiviral genome construct comprising a RNA polymerase I (Pol I) promoter.

A lentiviral genome construct of this disclosure may further comprise a Pol I termination signal.

A lentiviral genome construct of this disclosure may comprise or further comprise a 5′ long terminal repeat (5′ LTR) and/or a 3′ long terminal repeat (3′ LTR). The 5′ LTR may be positioned or located downstream of the RNA polymerase I (Pol I) promoter and the 3′ LTR may be positioned or located upstream of the Pol I termination signal.

The lentiviral genome construct may comprise or further comprise an insert, which insert is flanked by said 5′ and 3′ long terminal repeats. The insert may comprise a transgene.

In one teaching, a lentiviral genome construct of this disclosure may comprise:
a RNA polymerase I (Pol I) promoter;
a Pol I termination signal;
a 5′ long terminal repeat (5′ LTR);
a 3′ long terminal repeat (3′ LTR); and
an insert located or disposed between the 5′ and 3′ long terminal repeats;
   wherein, the 5′ LTR is positioned or located downstream of the RNA polymerase I (Pol I) promoter and the 3′ LTR is positioned or located upstream of the Pol I termination signal.

In one teaching, the disclosure provides a construct for use in an expression system, wherein the construct comprises:
an insert flanked by a 5′ long terminal repeat (5′ LTR) and a 3′ long terminal repeat (3′ LTR), wherein the 5′ LTR is downstream of a RNA polymerase I (Pol I) promoter and the 3′ LTR is upstream of a Pol I termination signal, wherein said insert comprises a transgene.

The term “expression system” as used herein typically comprises one or more constructs, such as one or more DNA constructs, that encode a protein, a peptide or an RNA when the construct(s) is/are provided to a cell. For example, an expression system may encompass a bacteria expression system, a yeast expression system or a mammalian expression system.

In one embodiment, there is provided a construct for use in producing recombinant retrovirus comprising an insert flanked by a 5′ long terminal repeat (5′ LTR) and a 3′ long terminal repeat (3′ LTR), wherein the 5′ LTR is downstream of an RNA polymerase I (Pol I) promoter and the 3′ LTR is upstream of a Pol I termination signal, wherein said insert comprises a transgene, and optionally, the construct further comprises a posttranscriptional response element downstream of said transgene and upstream of the Pol I termination signal, and further optionally, the construct comprises a retroviral packaging element and/or one or more nuclear export component(s) upstream of said insert

In one embodiment, a polyA signal is provided immediately (typically 20 to 200 bp, such as 40 to 100 bp downstream of the end of the gene coding sequence) after the transgene. Inclusion of strong polyA signals are well known to increase expression of genes, however are not generally included in current lentivirus designs as they would terminate Pol II transcription ahead of the 3′LTR.

By extension of the above, transgene expression systems that express two or more transgenes, typically currently use a cleavable peptide linker, to separate each transgene, but total transgene expression is under the control of a single promoter. However, in some cases it may be desirable to have the different transgenes under different promoter control. The present disclosure permits the expression of dual or multiple complete transgene systems and each transgene may be under control of a separate promoter, e.g. promoter-transgene-polyA signal. The, or each transgene may be under control of a pol I or pol II promoter, for example.

Pol I transcripts are not normally processed by the same splicing mechanism as Pol II transcripts. However, introns can be useful, and in some cases essential, for expression levels of certain transgenes. Thus, in one embodiment, the, or each, transgene, may comprise one or more introns, as well as the transgene coding or exon sequence.

It should be noted that throughout this specification the terms “comprise” and/or “comprising” are used to denote that aspects and/or embodiments of the present disclosure “comprise” the noted features and as such, may also include other features. However, in the context of this disclosure, the terms “comprise” and “comprising” encompass embodiments in which the present disclosure “consists essentially of” the relevant features or “consists of” the relevant features.

In one application, the constructs of the present disclosure are intended for use in producing non-replicating recombinant retrovirus for cell and/or gene therapy applications. Retroviral manufacturing systems typically involve a transfection system comprising multiple vectors encoding different parts of a retroviral particle. For safety, vectors containing the packaging and envelope components are typically delivered to a host cell separately from the retroviral vector genome. As used herein, the term “retroviral vector genome” generally refers to the genetic material that is transcribed from the construct disclosed herein and sought to be delivered to a target cell by a retroviral particle for cell and/or gene therapy applications.

The present disclosure may also embrace host cells in which the various lentiviral components (or genetic elements encoding the same) are stably integrated into the host cell genome. It should be noted that the provision of a stable (lentiviral vector) producer cell line represents a significant economic benefit, especially as this may obviate the need to produce high quantities of plasmid. The location within the cell genome where the viral components become stably integrated may be important, as the relative levels of expression must be both high and balanced. However, one of skill will appreciate that integration process is somewhat random and so location is not pre-determined. Therefore, the outcome of a balanced and high expression is somewhat determined when selecting for particular clone(s). For example, clones in which viral elements have integrated into unfavourable locations should be screened out. In these embodiments, Pol I may at least be used to drive transcription of the lentiviral genome integrated into the cell DNA (genome).

For the avoidance of doubt, the term “a host cell” as used herein refers to a cell (typically in vitro) to which the construct of the present disclosure is delivered (e.g. using transfection methods) for the production of non-replicating recombinant retroviruses. A host cell may also be referred to as a host manufacturing cell or a manufacturing cell as it is the host cell that produces the recombinant retrovirus to be used in cell and/or gene therapy applications. The term “a target cell” refers to a cell to which the resulting recombinant retrovirus comprising a transgene is delivered (e.g. using transduction methods) for cell and/or gene therapy applications. Typically, the recombinant retroviruses of the present disclosure delivered to a target cell are non-replicating recombinant retroviruses.

In one embodiment, the host cell is a mammalian cell. In a further embodiment, the mammalian cell may be selected from a HEK 293 cell, HEK 6E cell, CHO cell, BHK21 cell, NSD20 cell, Sp2/0 cell, HT-1080 cell, PER.c6 cell, HKB-11 cell, CAP cell, HuH-7 cell, Jurkat cell, KS62 cell, PerC6 cell, HeLa cell, HOS cell, H9 cell or a derivative or functional equivalent thereof. In a yet further embodiment, the mammalian host cell is a HEK 293 cell, or derived from a HEK 293 cell. Such cells could be adherent cell lines (i.e. they grow in a single layer attached to a surface) or suspension adapted/non-adherent cell lines (i.e. they grow in suspension in a culture medium). In a yet further embodiment, the HEK 293 cell is a HEK 293T cell or a HEK 6E cell. The term “HEK 293 cell” refers to the Human Embryonic Kidney 293 cell line, which is commonly used in biotechnology. In particular, HEK 293T cells are commonly used for the production of various retroviral vectors. Other examples of suitable commercially available cell lines include T REX™ (Life Technologies) cell lines. In an alternative embodiment, an insect cell line may be used as the host cell, such as Sf9 and Sf21 and High Five.

The constructs of the present disclosure provide an insert, which is transcribed in a host cell to produce (single-stranded positive sense RNA) transcript(s) that may be packaged into a retroviral particle as a retroviral vector genome. Each single stranded positive sense RNA transcript may form a “retroviral vector genome” of the resulting non-replicating recombinant retrovirus. Generally speaking, the construct of the present disclosure is used with other constructs that encode the viral packaging and/or envelope components.

As used herein, the term “transgene” refers to a nucleic acid sequence that has been artificially introduced into a construct of the present disclosure. Preferably, a packaged retrovirus genome comprises the transgene within it. The transgene may be partly or entirely heterologous or is homologous to an endogenous gene of the target animal or cell into which it is introduced, but which is designed to be inserted, or is inserted, into the target genome in such a way to alter the genome of the cell into which it is inserted. In some instances, the transgene may not be similar to an endogenous gene and introduced from another cell type or different species. For example, a bacterial protein may be delivered to mammalian cells using the construct provided herein for gene editing of a target cell using a lentivirus as described herein. A transgene of the present disclosure may contain sequences for exogenous protein production in a target cell, but may alternatively be non-coding in the sense that it does not encode a protein or peptide. In some embodiments, the construct of the present disclosure may comprise more than one transgene. In some embodiments, the construct of the present disclosure may comprise a transgene comprising a nucleic acid sequence for exogenous protein production, and optionally further comprise a nucleic acid sequence that encodes gRNA, miRNA, siRNA, shRNA and/or lncRNA.

In one embodiment, the transgene comprises a nucleic acid sequence of interest, such as a sequence that encodes one or more gene(s) of interest.

In one embodiment, the insert may optionally further comprise one or more promoter(s) for the expression of the transgene(s).

As used herein, a “gene” may refer to a nucleic acid sequence that encodes a functional RNA, which in certain instances, encode an amino acid sequence (e.g. protein or peptide). In some embodiments, a gene may or may not comprise introns. Thus, it should be appreciated that the term “gene” encompasses open reading frames (ORFs). The term ORF refers to a nucleic acid sequence (or polynucleotide sequence) that encodes an RNA and/or amino acid sequence, but which lacks introns. Thus, the entire sequence of an ORF with the possible exception of the final termination codon, encodes an RNA and/or amino acid sequence. Accordingly, “one or more gene(s) of interest” of the present disclosure may comprise a gene that encodes an RNA, miRNA, protein or peptide intended for a therapeutic purpose, wherein the RNA, miRNA, protein or peptide may be a foreign protein/peptide or a mammalian protein/peptide. The “one or more gene(s) of interest” may be of use in correcting or abrogating a defective gene or transcript within a target animal or cell, for example. The “one or more gene(s) of interest” may be of use in expressing a receptor or chimeric receptor for presentation on the surface of a cell, such as a T cell, in order to provide a therapeutic effect by altering an immune response in a target animal or cell.

In order to promote transcription of the construct in the host cell, the construct of the present disclosure comprises an RNA polymerase I promoter, which lies upstream of the 5′ LTR. RNA polymerase I promoters are highly constitutively active promoters. The promoters recognized by RNA polymerase I are not well conserved in sequence from one species to another. However, they all have similar general architecture of the promoter as it consists of a core element surrounding the transcription start site, and an upstream promoter element, which is about 100 bp farther upstream. RNA polymerase I, which transcribes rRNA genes, binds to a promoter containing a core promoter element and an upstream control element (UCE).

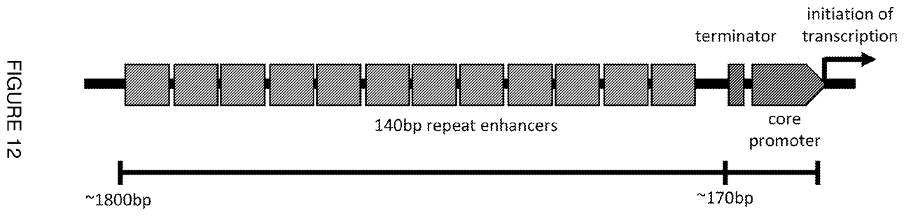

In eukaryotic cellular function, RNA polymerase I is responsible for the transcription of ribosmomal RNA (rRNA), an essential component of protein synthesis. The encoding sequences for rRNA are located in several repeated regions on the genome, with approximately 400 ribosomal DNA repeats within the human genome (Srivastava, A. K., & Schlessinger, D. (1991). Structure and organization of ribosomal DNA. Biochimie, 73 (6), 631-638). The RNA Pol I promoter drives transcription of the rRNA subunits 18S, 5.8S and 28S in a single transcript prior to processing into the individual subunits. Eukaryotic RNA polymerase I promoter consists of a core RNA Pol I promoter immediately upstream of the start of transcription. Upstream of the core promoter an RNA Pol I terminator sequence precedes a number of repeated sequences to approximately 1800 bp upstream of the initiation of transcription, which have been shown to enhance RNA Pol I transcription in cell-based assays (FIG. 12).

Therefore, for the production within human cell lines, such as HEK293, a human RNA Pol I promoter sequence is preferably used. In one embodiment, the RNA polymerase I promoter is derived from the same or related species as the host cell of interest. In one embodiment, RNA polymerase I (Pol I) promoter is a mammalian Pol I promoter. In a preferred embodiment, RNA polymerase I (Pol I) promoter is a human Pol I promoter. In one embodiment, the RNA polymerase-promoter may comprise the sequence TTTCGCTCCGAGTCGGCATTTTGGGCCGCCGGGTTATT (SEQ ID NO: 1). In some embodiments, the RNA polymerase promoter may comprise the sequence GGCCGGCCCCCTGCGTGTGGCACGGGCGGCCGGGAGGGCGTCCCCGGCCCGGCGCTGCTCCCGCGTGT GTCCTGGGGTTGACCAGAGGGCCCCGGGCGCTCCGTGTGTGGCTGCGATGGTGGCGTTTTTGGGGACA GGTGTCCGTGTCGCGCGTCGCCTGGGCCGGCGGCGTGGTCGGTGACGCGACCTCCCGGCCCCGGGGGA GGTATATCTTTCGCTCCGAGTCGGCATTTTGGGCCGCCGGGTTATT (SEQ ID NO: 2), or a fragment thereof. A fragment may comprise a portion of a reference sequence, such as 50%, 60%, 70% 80%, 85%, 90%, 95%, or 99% of the length of a sequence provided herein. Optionally, or in addition, the RNA polymerase I (Pol I) promoter may further comprise one or more Pol I enhancer element(s). For instance, Pol I enhancer element(s) may include a number of upstream repeat 140 bp elements as described in Pikaard et al 1990, [Pikaard, C. S., Pape, L. K., Henderson, S. L., Ryan, K., Paalman, M. H., Lopata, M. A., . . . & Sollner-Webb, B. (1990). Enhancers for RNA polymerase I in mouse ribosomal DNA. Molecular and cellular biology, 10 (9), 4816-4825.], or proximal downstream control elements as described by Haltiner et al (1985) [Haltiner, M. M., Smale, S. T., & Tjian, R. (1986). Two distinct promoter elements in the human rRNA gene identified by linker scanning mutagenesis. Molecular and cellular biology, 6 (1), 227-235.]. In addition, eukaryotic RNA Pol II enhancer elements may be used as described by Lorch et al (1990) [Lorch, Y., Lue, N. F., & Kornberg, R. D. (1990). Interchangeable RNA polymerase I and II enhancers. Proceedings of the National Academy of Sciences, 87 (21), 8202-8206.] In addition, or alternatively, the RNA polymerase I promoter may comprise a downstream sequence, which promotes or increases transcription.

In mice, the termination RNA polymerase I transcription is mediated through a series of 10 to 18 nucleotide repeat elements (termed Sal box of sequence AGGTCGACCAGTACTCCG). Proximate to the Sal box there are pyrimidine rich tracts (T or C) of at least 10 nucleotides in length. In cells, the first Sal box has been demonstrated as sufficient for the termination of RNA Pol I transcription (Grummt, I., Maier, U., Ohrlein, A., Hassouna, N., & Bachellerie, J. P. (1985). Transcription of mouse rDNA terminates downstream of the 3′ end of 28S RNA and involves interaction of factors with repeated sequences in the 3′ spacer. Cell, 43 (3), 801-810).

A mouse terminator sequence containing a combination of the Sal box and pyrimidine tracts both upstream and downstream have been shown to be sufficient to terminate RNA Pol I transcription in minigene experiments in both mouse and human cells (Hadjiolova, K. V., Normann, A., Cavaillé, J., Soupène, E., Mazan, S., Hadjiolov, A. A., & Bachellerie, J. P. (1994). Processing of truncated mouse or human rRNA transcribed from ribosomal minigenes transfected into mouse cells. Molecular and Cellular Biology, 14 (6), 4044-4056.)

This RNA Pol I termination signal would therefore generally be required downstream of the 3′ LTR. This termination signal may be much less species specific, and as such, various mammalian termination signals may be used. In one embodiment, a mouse termination signal or a human termination signal may be used (Németh et al. (2013), Biochimica et Biophysica Acta (BBA)-Gene Regulatory Mechanisms, 1829 (3-4), 306-317; Normann et al. (1994), Molecular and Cellular Biology, 14 (6), 4044-4056). In some embodiments, a termination signal derived from a mouse termination signal and/or a human termination signal may be used. In one embodiment, the RNA polymerase I termination signal may comprise the sequence (SEQ ID NO: 3):

TCCCCCCCAACTTCGGAGGTCGACCAGTACTCCGGGCGACAC
TTTGTTTTTTTTTTTTCCCCCGATGCTGGAGGTCGA

In the sequence provided above (SEQ ID NO: 3), the sequence of mouse RNA Pol I terminator with Sal box is underlined and pyrimidine tracts are shown in bold.

In some embodiments, the terminal sequence of the RNA polymerase I promoter may comprise the sequence TTATT, TTATAT or a derivative thereof. The terminal sequence of the RNA polymerase I promoter comprising TTATT, TTATAT or a derivative thereof may further comprise 1, 2, 3, 4, 5, 6, 7, 8, 9, 10, 11, 12, 13, 14, 15, 20, 25, 30, 35, 40, 45, 50. 55. 60, 65, 70, 75 or 80 additional nucleotides at the 5′ or 3′ end of the terminal sequence.

In some embodiments, in addition to the terminal sequence of the RNA polymerase I promoter TTATT, TTATAT or a derivative thereof, the construct as disclosed herein may optionally comprise the sequence GCTGACACGCTGTCCTCT (SEQ ID NO: 4) downstream of said terminal sequence of the RNA polymerase I promoter.

In some embodiments, the RNA polymerase I promoter of the construct of the present disclosure may comprise the sequence as follows:

(SEQ ID NO: 5)
GAGGGGCTGCGTTTTCGGCCTCGGGAAGAGCTTCTCGACTCACGG
TTTCGCTTTCGCGGTCCACGGGCCGCCCTGCCAGCCGGATCTGTC
TCGCTGACGTCCGCGGCGGTTGTCGGGCTCCATCTGGCGGCCGCT
TTGAGATCGTGCTCTCGGCTTCCGGAGCTGCGGTGGCAGCTGCCG
AGGGAGGGGACCGTCCCCGCTGTGAGCTAGGCAGAGCTCCGGAAA
GCCCGCGGTCGTCAGCCCGGCTGGCCCGGTGGCGCCAGAGCTGTG
GCGCGTCGCTTGTGAGTCACAGCTCTGGCGTGCAGGTTTATGTGG
GGGAGAGGCTGTCGCTGCGCTTCTGGGCCCGCGGCGGGCGTGGGG
CTGCCCGGGCCGGTCGACCAGCGCGCCGTAGCTCCCGAGGCCCGA
GCCGCGACCCGCGGGGACCCGCCGCGCGTGGCGCGGGAGGCTGGG
GACGCCCTTCCCGGCCCGGTCGCGGGTCCGCGCTCATCCTGGCCG
TCTGAGGCGGCGGCCGAATTCGTTTCCGAGTCCCCGTGGGGAGCC
GGGGACCGTCCCGCCCCCGTCCCCCGGGTGCCGGGGAGCGGTCCC
TCTGCCGCGATCCTTTCTGGCGAGTCCCCGTGCGGAGTCGGAGAG
CGCTCCCTGAGCGCGCGTGCGGCCCGAGAGGTCGCGCCTGGCCGG
CCTTCGGTCCCTCGTGTGTCCCGGTCGTAGGAGGGGCCGGCCGAA
AATGCTTCCGGCTCCCGCTCTGGAGACACGGGCCGGCCCCCTGCG
TGTGGCACGGGCGGCCGGGAGGGCGTCCCCGGCCCGGCGCTGCTC
CCGCGTGTGTCCTGGGGTTGACCAGAGGGCCCCGGGCGCTCCGTG
TGTGGCTGCGATGGTGGCGTTTTTGGGGACAGGTGTCCGTGTCGC
GCGTCGCCTGGGCCGGCGGCGTGGTCGGTGACGCGACCTCCCGGC
CCCGGGGGAGGTATATCTTTCGCTCCGAGTCGGCATTTTGGGCCG
CCGGGTTATT

A commonly adopted approach in the art for the production of recombinant viruses relies on RNA polymerase II promoter(s) for recombinant virus synthesis. These transcripts are produced with a poly (A) tail which dogmatically would allow for RNA splicing to occur, as well as direct them to the protein production machinery within the host cell, reducing their availability for viral packaging. The constructs of the present disclosure employ the use of a RNA polymerase I termination signal rather than a polyadenylation signal and thus provides an advantage of bypassing the processing step prior to packaging into the viral particle. In one embodiment, the construct of the present disclosure does not comprise a polyadenylation signal.

In order to induce integration of the transgene into a target animal or cell genome, long terminal repeat (LTR) regions are typically required for integration into the target genome. Long Terminal Repeats (LTRs): The basic structure of a retrovirus genome comprises a 5′-LTR and a 3′-LTR, between or within which are located the genes required for retroviral production. The LTRs are required for retroviral integration and transcription. They can also act as promoter sequences to control the expression of the retroviral genes (i.e. they are cis-acting sequences). In some instances, the construct of the present disclosure may not comprise a cis-acting polyadenylation sequence within the 5′ LTR region. The LTRs are composed of three sub-regions designated U3, R, U5: U3 is derived from the sequence unique to the 3′ end of the RNA; R is derived from a sequence repeated at both ends of the RNA; and U5 is derived from the sequence unique to the 5′ end of the RNA. Therefore, in accordance with the disclosure, the constructs comprise a 5′- and 3′-LTR. In a further embodiment, the U5 region of the 3′ LTR can be deleted and replaced with a non-HIV-1 polyA tail (see Hanawa et al. (2002) Mol. Ther. 5 (3): 242-51).

In an embodiment of the disclosure, the LTRs present in the nucleic acid vectors of the disclosure can be self-inactivating LTRs (termed SIN LTRs). In order to address safety concerns relating to the generation of replication-competent virus, a self-inactivating (SIN) vector has been developed by deleting a section in the U3 region of the 3 LTR, which includes the TATA box and binding sites for transcription factors Spl and NF-κB (see Miyoshi et al. (1998) J. Virol. 72 (10): 8150-7). The deletion is transferred to the 5′ LTR after reverse transcription and integration in infected cells, which results in the transcriptional inactivation of the LTR. This is known as a self-inactivating lentiviral-based vector system, which may be encompassed in the present disclosure.

The 5′ and 3′ LTRs of the construct disclosed herein flank the transgene (also referred to as “payload”). The 5′ and 3′ LTRs of the construct of the present disclosure may be derived from a viral genome. In one embodiment, the 5′ LTR and/or 3′ LTR sequences are derived from a retroviral genome, such as sequences disclosed in Bulcha et al. (2021) [1], for example. In a preferred embodiment, the 5′ LTR and/or 3′ LTR sequences are derived from a lentiviral genome. Alternatively, the 5′ LTR and/or 3′ LTR sequences may be derived from a mammalian sequence, such as a human 5′ LTR and/or 3′ LTR sequence.

In one embodiment, the 5′ LTR nucleic acid sequence of the construct of the present disclosure may comprise the following sequence (SEQ ID NO: 6):

GGGTCTCTCTGGTTAGACCAGATCTGAGCCTGGGAGCTCTCTGGC
TAACTAGGGAACCCACTGCTTAAGCCTCAATAAAGCTTGCCTTGA
GTGCTTCAAGTAGTGTGTGCCCGTCTGTTGTGTGACTCTGGTAAC
TAGAGATCCCTCAGACCCTTTTAGTCAGTGTGGAAAATCTCTAGC
A

In one embodiment, the 3′ LTR nucleic acid sequence of the construct of the present disclosure may comprise the following sequence (SEQ ID NO: 7):

TGGAAGGGCTAGCTCACTCCCAACGAAGACAAGATCTGCTTTTTG
CTTGTACTGGGTCTCTCTGGTTAGACCAGATCTGAGCCTGGGAGC
TCTCTGGCTAACTAGGGAACCCACTGCTTAAGCCTCAATAAAGCT
TGCCTTGAGTGCTTCAAGTAGTGTGTGCCCGTCTGTTGTGTGACT
CTGGTAACTAGAGATCCCTCAGACCCTTTTAGTCAGTGTGGAAAA
TCTCTAGCA

In addition to the transgene, the insert of the construct disclosed herein may optionally comprise one or more viral components necessary for the assembly of a functional or infectious viral particle. For instance, the construct may comprise sequences that promote the retroviral vector genome to be packaged into a viral particle following transfection of the host cell.

Thus, in a further embodiment, the one or more element(s) of said insert may optionally comprise one or more viral packaging component(s) and/or one or more nuclear export component(s) (Milone and O'Doherty (2018), Leukemia, 32 (7): 1529-1541; Dull T et. al. (1998), J. Virol. 72 (11): 8463-8471. A Third Generation Lentivirus Vector with a Conditional Packaging System). In a preferred embodiment, the viral packaging component(s) and/or nuclear export component(s) of said insert lies downstream of 5′ LTR and upstream of the transgene. ψ: Encapsidation of the retroviral RNAs occurs by virtue of a y (psi) sequence located at the 5′ end of the retroviral genome. It is also well known in the art that sequences downstream of the psi sequence and extending into the gag coding region are involved in efficient retroviral vector production (see Cui et al. (1999) J. Virol. 73 (7): 6171-6176). In one embodiment, the nucleic acid vector additionally comprises a ψ (psi) sequence. In some embodiments, the one or more viral packaging component(s) may comprise a retroviral packaging element, such as HIV-1 ψ, and the one or more nuclear export component(s) may comprise a Rev response element (RRE). In one embodiment, the RRE may comprise the sequence (SEQ ID NO: 8):

ATGGCAGGAAGAAGCGGAGACAGCGACGAAGACCTCCTCAAGGCA
GTCAGACTCATCAAGTTTCTCTATCAAAGCAACCCACCTCCCAAT
CCCGAGGGGACCCGACAGGCCCGAAGGAATAGAAGAAGAAGGTGG
AGAGAGAGACAGAGACAGATCCATTCGATTAGTGAACGGATCCTT
AGCACTTATCTGGGACGATCTGCGGAGCCTGTGCCTCTTCAGCTA
CCACCGCTTGAGAGACTTACTCTTGATTGTAACGAGGATTGTGGA
ACTTCTGGGACGCAGGGGGTGGGAAGCCCTCAAATATTGGTGGAA
TCTCCTACAATATTGGAGTCAGGAGCTAAAGAATAG

Optionally, the construct of the present disclosure may additionally comprise one or more export and/or stabilising motif(s), which facilitate the stability and/or nuclear export of the transcript.

One of skill will recognise that for the expression of the transgene in the target cell, the transgene is likely to require a promoter operably or functionally linked to the gene of interest.

As used herein, the term “functionally linked” refers to two or more nucleic acid sequences, or partial sequences, which are positioned such that they functionally interact to perform their intended functions. For example, a promoter is functionally linked to a nucleic acid sequence if it is able to control or modulate transcription of the linked nucleic acid sequence typically in the cis position. Generally, but not necessarily, functionally linked nucleic acid sequences are close together. Although a functionally linked promoter is generally located upstream of the coding sequence, it may not necessarily be close to the coding sequence.

In one embodiment, the one or more promoter(s) for the expression of the transgene may comprise a Pol I or a Pol II promoter. For instance, promoters that may be used for the expression of a transgene are discussed in Milone, M. C., & O'Doherty, U. (2018), Clinical use of lentiviral vectors. Leukemia, 32 (7), 1529-1541. In some embodiments, an endogenous promoter may be used for targeting expression in specific tissue types, such as the liver, for example. In some examples, tissue specific Pol II promoters may be used to target expression of one or more genes to preferred cell types (see Frecha, C., Szecsi, J., Cosset, F. L., & Verhoeyen, E. (2008). Strategies for targeting lentiviral vectors. Current gene therapy, 8 (6), 449-460).

In one embodiment, the one or more promoter(s) for the expression of the transgene may be a Pol II promoter(s), such as CMV, RSV, SV40, EFS (EF1a short), Ubc promoter, or SFFV promoter, for example.

In addition, or alternatively, in some embodiments, wherein the transgene of the construct comprises a sequence that encodes a naturally occurring or synthetic non-coding RNA, such as gRNA, the construct may comprise a Pol I promoter for the expression of said non-coding RNA. In some embodiments, the transgene of the construct may comprise a Pol II promoter for the expression a Cas protein and a Pol I promoter for the expression of gRNA.

The synthesis of recombinant viruses typically involves the delivery of vectors encoding the viral components to a host cell. As such, in a preferred embodiment, the construct of the present disclosure is typically provided as a vector. Any suitable vector known in the art may be employed in recombinant virus production, such as artificial chromosomes (e.g. bacterial or yeast), plasmids, cosmids, or phagemids, for example. One of skill will be familiar with various vectors that may be used to transfect a host cell for the replication of the viral genome and the synthesis of viral proteins. In one embodiment, the vector may be a plasmid vector. For example, the construct of the present disclosure may be inserted into a pEX-K168 vector. In other examples the construct may be inserted into a vector such as pMX, pMA, pMK, pcDNA 3.1 (+), pcDNA3.3-TOPO, pcDNA3.4-TOPO, pDONR221, pET100/D-TOPO, pET151/D-TOPO, pFastBac I, pRSET_A, pYes2.1V5-His TOPO, pBAD_His_B, pBAD_His_C, pBADHis_A, pcDNA3.1_Zeo, pcDNA3.1+Hygro, pcDNA5_FRT, pDONR_zeo, pFastBacHTA, pFastBacHTb, pFastBacHTC, pPICZalphaA, pPICZalphaB, pPICZalphaC, pRSET_B, pRSET_C or pYES2. A suitable vector for use in producing recombinant viruses of the present disclosure is typically capable of accommodating an insert with a size of up to1 kbp, 2 kbp, 3 kbp, 4 kbp, 5 kbp, 6 kbp, 7 kbp, 8 kbp, 9 kbp, 10 kbp, 15 kbp, 20 kbp or 25 kbp.

In the present disclosure, the construct or the vector of the present disclosure may comprise some of the retroviral sequences that occur in a wild type retroviral genome. In a preferred embodiment, the insert comprising the transgene of the present disclosure may replace most of the viral sequences to form a retroviral vector genome so long as the transcript resulting from the construct disclosed herein is capable of being packaged into a retroviral particle.

In one embodiment, the construct of the present disclosure may be used to produce a recombinant lentivirus. Lentiviruses (a subtype of retrovirus) provide the advantage of being capable of transducing dividing and non-dividing cells, leading to sustained expression of the gene. Suitable lentiviruses from which a portion of the retroviral vector genome may be derived include, but are not limited to, those based on Human Immunodeficiency Virus (HIV-1), HIV-2, EIAV, feline immunodeficiency virus (FIV), Simian Immunodeficiency virus (SIV) and maedi/visna virus, for example. As used herein, “a portion of” refers to at least 5%, 10%, 20%, 30%, 40%, 50%, 60%, 70%, 80%, 90% or 95% of the full length of the sequence of interest. In one embodiment, the construct is for use in producing lentivirus.

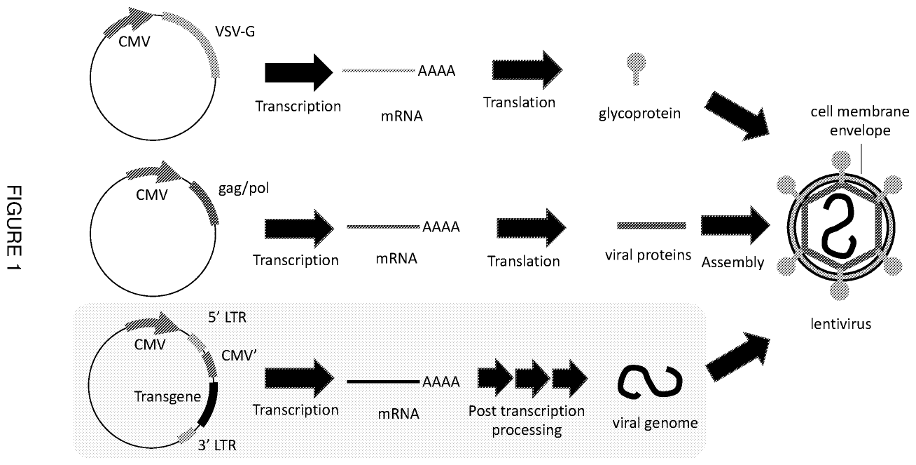

Various methods of producing recombinant retroviruses will be evident to a skilled person, such as using transfection methods widely adopted in the art. Current manufacturing practice typically uses the well-characterised human embryonic kidney cells (HEK293, HEK293T) in the production of the lentivirus. Alternative cell lines that may be used in the production of lentivirus include CHO cells, BHK21 cells, murine myeloma cells (NS0 and/or Sp2/0), HT-1080 cells, PER.C6 cells, HKB-11 cells, CAP cells, HuH-7 cells, Jurkat human cells lines, Sf9 cells, Sf21 and High Five cells, for example. A triple (sometimes quadruple) transfection approach is used where the packaging, envelope and viral genome plasmids are transfected into the manufacturing cells in order to produce the lentivirus (FIG. 1). The envelope protein, VSV-G can be substituted for other proteins to allow targeted infection of other cell types depending on the application. For instance, the envelope protein may alternatively be selected from murine leukemia virus (MULV), the gibbon ape leukemia virus (GALV), the feline endogenous RD114 retrovirus, Moloney MULV 4070A, Moloney MULV strain 10A1, rabies virus glycoprotein, and/or the measles virus hemagglutinin and fusion glycoprotein, for example. However, without this VSV-G, or other similar envelope protein, the virus cannot infect a target cell. The packaging components Gag, Pol and Rev are also essential structural and functional viral components. These elements are all typically manufactured as protein components within the manufacturing host cell line.

As such, in a further aspect, the present disclosure provides a system for producing recombinant lentivirus, wherein the system comprises the construct or the vector comprising the construct of the present disclosure and optionally, one or more vector(s) comprising:
one or more viral genome packaging component(s); and/or one or more viral envelope protein(s).

In one embodiment, the one or more viral genome packaging component(s) may comprise Gag, Pol and/or Rev. In one embodiment, the one or more viral envelope protein(s) may comprise Env, such as VSV-G.

Gag/pol: The expression of gag and pol genes relies on a translational frameshift between gag and pol. Both are polyproteins, which are cleaved during maturation. The major structural matrix, capsid, and nucleocapsid proteins of the retroviral vector are encoded by gag. The pol gene codes for the retroviral enzymes: i) reverse transcriptase, essential for reverse transcription of the retroviral RNA genome to double stranded DNA, ii) integrase, which enables the integration of the retroviral DNA genome into a host cell chromosome, and iii) protease, that cleaves the synthesized polyprotein in order to produce the mature and functional proteins of the retrovirus. In one embodiment, the retroviral nucleic acid sequence encoding the gag and pol proteins is derived from the HIV-1 HXB2 sequence, which is available at Genome Accession No. K03455, for example from base pairs 790-5105.

Rev: The auxiliary gene rev (“regulator of virion”) encodes an accessory protein which binds to the Rev Response element (RRE) and facilitates the export of retroviral transcripts. The gene's protein product allows fragments of retroviral mRNA that contain the Rev Responsive element (RRE) to be exported from the nucleus to the cytoplasm. The RRE sequence is predicted to form a complex folded structure. This particular role of rev reflects a tight coupling of the splicing and nuclear export steps. In one embodiment, nucleic acid vector comprises an RRE sequence. In a further embodiment, the RRE sequence is derived from HIV-1 HXB2 sequence, which is available at Genome Accession No. K03455, for example from base pairs 7622 to 8479, or 7769 to 8146, in particular base pairs 7622 to 8479.

Rev binds to RRE and facilitates the export of singly spliced (env, vif, vpr and vpu) or non-spliced (gag, pol and genomic RNA) viral transcripts, thus leading to downstream events like gene translation and packaging (see Suhasini and Reddy (2009) Curr. HIV Res. 7 (1): 91-100). In one embodiment, the nucleic acid vector additionally comprises the auxiliary gene rev or an analogous gene thereto (i.e. from other retroviruses or a functionally analogous system). Inclusion of the rev gene ensures efficient export of RNA transcripts of the retroviral vector genome from the nucleus to the cytoplasm, especially if an RRE element is also included on the transcript to be transported. In a further embodiment, the rev gene comprises at least 60% sequence identity, such as at least 70% sequence identity to base pairs 970 to 1320 of Genome Accession No. MI 1840 (i.e. HIV-1 clone 12 cDNA, the HIVPCV12 locus). In an alternative embodiment, the rev gene comprises at least 60% sequence identity, such as at least 70%, 80%, 90% or 100% sequence identity to base pairs 5970 to 6040 and 8379 to 8653 of Genome Accession No. K03455.1 (i.e. Human immunodeficiency virus type 1, HXB2).

Env: The env (“envelope”) gene codes for the surface and transmembrane components of the retroviral envelope (e.g. glycoproteins gpl20 and gp41 of HIV-1) and is involved in retroviral-cell membrane fusion. In order to broaden the retroviral vector's tissue tropism, the retroviral vectors described herein may be pseudotyped with an envelope protein from another virus. Pseudotyping refers to the process whereby the host cell range of retroviral vectors, including lentiviral vectors, can be expanded or altered by changing the glycoproteins (GPs) on the retroviral vector particles (e.g. by using GPs obtained from or derived from other enveloped viruses or using synthetic/artificial GPs). The most commonly used glycoprotein for pseudotyping retroviral vectors is the Vesicular stomatitis virus GP (VSVg), due to its broad tropism and high vector particle stability. However, it will be understood by the skilled person that other glycoproteins may be used for pseudotyping (see Cronin et al. (2005) Curr. Gene Ther. 5 (4): 387-398, herein incorporated by reference in its entirety). The choice of virus used for pseudotyping may also depend on the type of cell and/or organ to be targeted because some pseudotypes have been shown to have tissue-type preferences.

In one embodiment, the env protein or a functional substitute thereof is obtained from or derived from a virus selected from a Vesiculovirus (e.g. Vesicular stomatitis virus), Lyssavirus (e.g. Rabies virus, Mokola virus), Arenavirus (e.g. Lymphocytic choriomeningitis virus (LCMV)), Alphavirus (e.g. Ross River virus (RRV), Sindbis virus, Semliki Forest virus (SFV), Venezuelan equine encephalitis virus), Filovirus (e.g. Ebola virus Reston, Ebola virus Zaire, Lassa virus), Alpharetrovirus (e.g. Avian leukosis virus (ALV)), Betaretrovirus (e.g. Jaagsiekte sheep retrovirus (JSRV)), Gammaretrovirus (e.g. Moloney Murine leukaemia virus (MLV), Gibbon ape leukaemia virus (GALV), Feline endogenous retrovirus (RD114)), Deltaretrovirus (e.g. Human T-lympho trophic virus 1 (HTLV-1)), Spumavirus (e.g. Human foamy virus), Lentivirus (e.g. Maedi-visna virus (MVV)), Coronavirus (e.g. SARS-COV), Respirovirus (e.g. Sendai virus, Respiratory syncytia virus (RSV)), Hepacivirus (e.g. Hepatitis C virus (HCV)), Influenzavirus (e.g. Influenza virus A) and Nucleopolyhedrovirus (e.g. Autographa californica multiple nucleopolyhedrovirus (AcMNPV)). In a further embodiment, the env protein or a functional substitute thereof is obtained from or derived from Vesicular stomatitis virus. In this embodiment, the Vesicular stomatitis virus glycoprotein (VSVg) protein may be used which enables the retroviral particles to infect a broader host cell range and eliminates the chances of recombination to produce wild-type envelope proteins. In a further embodiment, the retroviral nucleic acid sequence encoding the env protein or a functional substitute thereof, is derived from the sequence available at Genome Accession No. J02428.1, for example from base pairs 3071 to 4720.

The structural genes described herein are common to all retroviruses. Further auxiliary genes may be found in different types of retrovirus. For example, lenti viruses, such as HIV-1, contain six further auxiliary genes known as rev, vif, vpu, vpr, nef and tat. Other retroviruses may have auxiliary genes which are analogous to the genes described herein, however they may not have always been given the same name as in the literature. References such as Tomonaga and Mikami (1996) J. Gen. Virol. 77 (Pt 8): 1611-1621 describe various retrovirus auxiliary genes.

Auxiliary genes are thought to play a role in retroviral replication and pathogenesis, therefore many current viral vector production systems do not include some of these genes. The exception is rev, which is usually present or a system analogous to the rev/RRE system is potentially used. Therefore, in one embodiment, the nucleic acid sequences encoding one or more of the auxiliary genes vpr, vif, vpu, tat and ne or analogous auxiliary genes, are disrupted such that said auxiliary genes are removed from the RNA genome of the retroviral vector particle or are incapable of encoding functional auxiliary proteins. In a further embodiment, at least two or more, three or more, four or more, or all of the auxiliary genes vpr, vif, vpu, tat and nef or analogous auxiliary genes, are disrupted such that said auxiliary genes are removed from the RNA genome of the retroviral vector particle or are incapable of encoding functional auxiliary proteins. Removal of the functional auxiliary gene may not require removal of the whole gene; removal of a part of the gene or disruption of the gene will be sufficient. It will be understood that the nucleic acid sequences encoding the replication defective retroviral vector particle may be the same as, or derived from, the wild-type genes of the retrovirus upon which the retroviral vector particle is based, i.e. the sequences may be genetically or otherwise altered versions of sequences contained in the wild-type virus. Therefore, the retroviral genes incorporated into the nucleic acid vectors or host cell genomes, may also refer to codon-optimised versions of the wild-type genes.

In a further embodiment, the one or more vectors may additionally comprise one or more nuclear export element(s) and/or one or more stabilising motif(s). In one embodiment, the one or more nuclear export element(s) may comprise Rev response element (RRE).

In order to increase the safety of the system for producing recombinant retroviruses, the construct comprising the viral vector genome may be delivered to the host cell in a separate construct or vector from the packaging and envelope components. In a preferred embodiment, the one or more optional vector(s) comprising one or more viral genome packaging component(s) and/or one or more viral envelope protein are delivered to the host cell in one or more separate vector(s) from the viral vector genome.

In a further aspect, there is provided a method for producing recombinant lentivirus, wherein the method comprises:
a) transfecting a host cell by contacting the said host cell with the construct as disclosed herein, and one or more vector(s) encoding one or more additional viral components;
b) culturing the host cell under conditions in which the non-replicative retroviral vector particle is produced; and
c) isolating said non-replicative retroviral vector particles.

In one embodiment, the one or more additional viral components are selected from:
(i) one or more packaging component(s); and/or
(ii) one or more envelope protein(s).

In one embodiment, one or more packaging component(s) comprise Gag, Pol and/or Rev, and the envelope protein comprises VSV-G.

In one embodiment, the method of the present disclosure may further comprise contacting the resulting recombinant lentivirus with a target cell in vitro, in vivo or ex vivo to induce integration of at least a portion of the construct of the present disclosure into the genome of the target cell. In some embodiments wherein the method comprises contacting the resulting recombinant lentivirus with a target cell in vitro or ex vivo, the method may optionally further comprise providing said target cell to a subject.

In one embodiment, the resulting recombinant lentivirus in accordance with any of the foregoing methods disclosed herein may be used as a medicament or in therapy. In an alternative embodiment, the resulting recombinant lentivirus may be used in gene therapy.

A method for producing recombinant lentivirus may comprise the provision of a cell (or cell line) in which the various lentiviral components (including the gag/pol, VSV-G and genome components) are stably integrated into the cell genome for expression, in such a cell at least the (lentiviral) genome component may comprise a RNA polymerase I (Pol I) promoter.

It is envisaged that any of the constructs or vectors comprising said construct of the present disclosure may be for use as a medicament or in therapy, such as gene therapy. For example, gene therapy may involve replacing a defective gene in a target cell with a transgene in order to restore expression levels of a protein of interest. In another example, the resulting recombinant retroviruses as disclosed herein may be used in cancer therapeutics, wherein the recombinant retroviruses are used to induce the expression of chimeric antigen receptors in the patient's T-cells.

In a further teaching, the present disclosure may further relate to a composition comprising the construct or vector as described herein, formulated with at least one acceptable excipient thereof. The composition comprising the construct or the vector of the present disclosure may be for use with other plasmid(s), such as a plasmid encoding the viral packaging In one embodiment, an acceptable excipient may comprise water, saline (e.g. phosphate-buffered saline), calcium phosphate, diethylaminoethyl (DEAE)-dextran, cationic lipids, human serum albumin, dextrose, trehalose, sucrose, mannitol, sorbitol, polysorbate 20, polysorbate 80, glycerol, ethanol, polyethylene glycol, or the like and combinations thereof. In some embodiments, the composition may comprise one or more excipients that promote cellular uptake of the construct or vector. Alternatively, or in addition, any suitable transfection buffers known in the art may be used to formulate the composition.

### DETAILED DESCRIPTION

The present disclosure is described with reference to the following figures, which show:

FIG. 1. A cartoon schematic of a triple transfection system where the packaging plasmid (typically encodes Gag/Pol) and the envelope plasmid (usually encodes VSV-G but may comprise an alternative envelope component) are co-transfected into a manufacturing cell line with the viral genome or transfer plasmid encoding the insert of interest. In this system all viral elements are driven by RNA Pol II promoters, such as CMV.

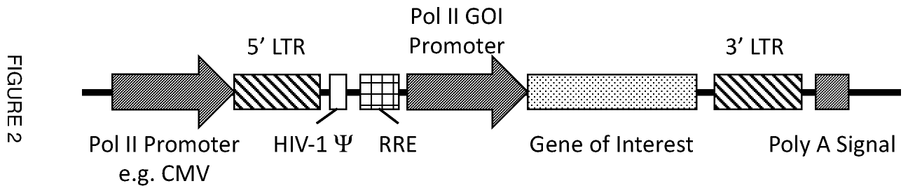

FIG. 2. Exemplar viral genome plasmid, excluding plasmid backbone elements. Pol II promoter (grey) along with 5′ and 3′ LTRs (stipes), the viral packaging and nuclear export elements HIV-1 Y and RRE respectively (white and hatched), internal gene of interest (GOI) Pol II promoter (grey), gene of interest (dots) and the terminating poly (A) signal (grey).

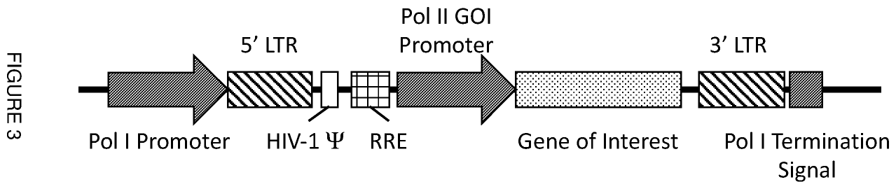

FIG. 3. Proposed viral genome plasmid structure using RNA Pol I. The elements between the 5′ LTR and 3′ LTR (stripes) remain unaltered as in FIG. 2. The promoter upstream of 5′ LTR is replaced with a species specific RNA Pol I promoter sequence (grey) dependent on the production cell line. For mammalian production, a mouse Pol I termination signal (grey) is required to terminate transcription.

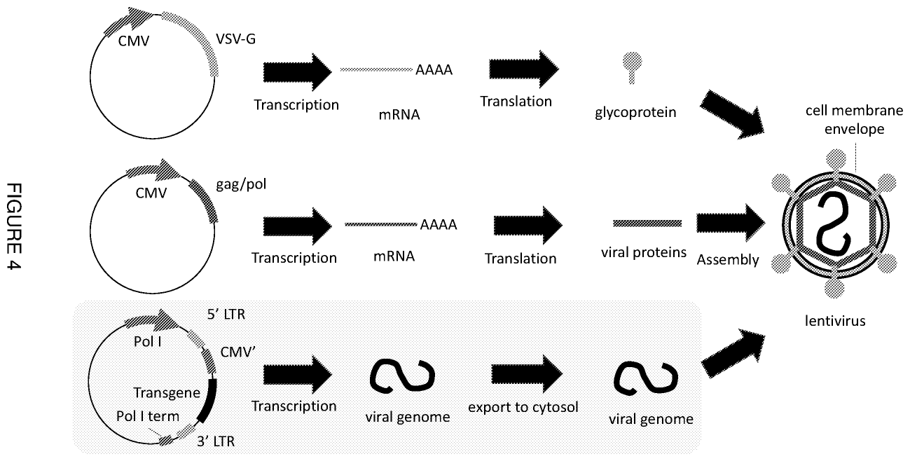

FIG. 4. Cartoon schematic of a triple transfection system where an RNA Pol I promoter drives viral genome production. The packaging plasmid (encoding Gag/Pol) and the envelope plasmid (encoding an envelope protein, such as VSV-G) remain unchanged.

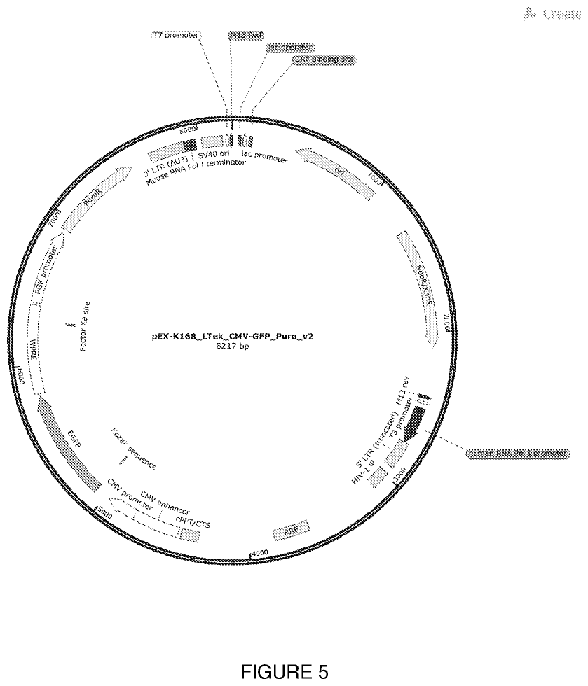

FIG. 5. Diagram of Pol I test genome plasmid named here as pEX-K168_LTek_CMV-GFP_Puro_v2. The RSV promoter has been replaced by 249 bp of the human RNA Pol I promoter sequence and the SV40 poly (A) signal replaced with the mouse RNA Pol I terminator sequence. The terminator sequence was directly abutted to the 3′ LTR without any spacing sequence.

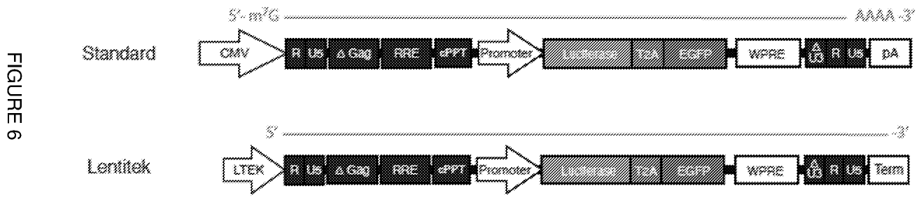

FIG. 6. Diagram of the transfer plasmid maps. The standard plasmid is based on the widely used pCCL system with a Luciferase/GFP fusion reporter transgene. The Lentitek plasmid has had the Pol I promoter (LTEK) and Pol I terminator (Term) inserted as described above. The promoter driving the Luciferase/GFP transgene is an endogenous liver promoter.

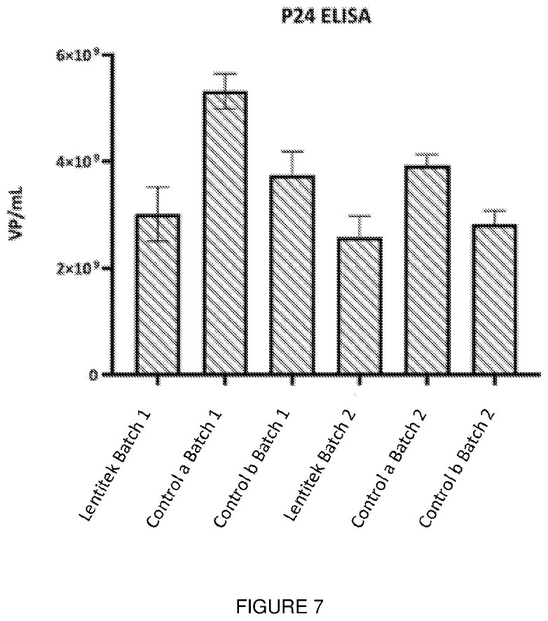

FIG. 7. p24 ELISA results. Quantification of lentivirus titre by p24. Pol I (Lentitek Batch 1 or 2) are less than, but of a similar magnitude to Pol II control plasmids.

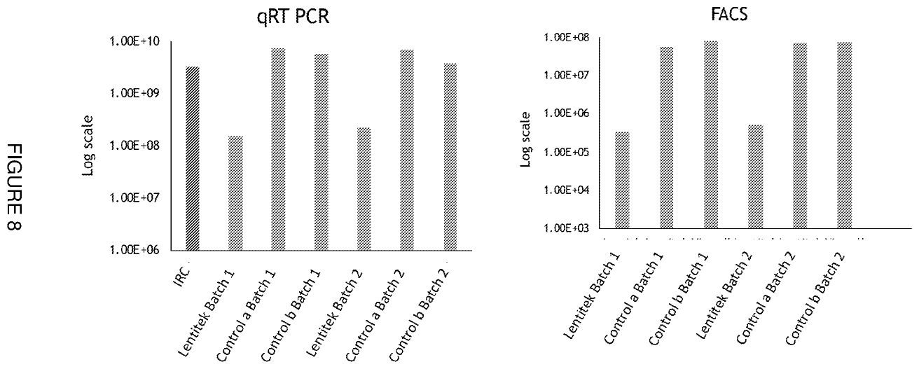

FIG. 8. FACS and qPCR titres. Left panel shows the qPCR against Psi of the lentivirus genome. Right panel shows the FACS data for GFP expression in transformed cells. In both cases the Pol I driven titre (Lentitek 1 and 2) are approximately 2 logs lower.

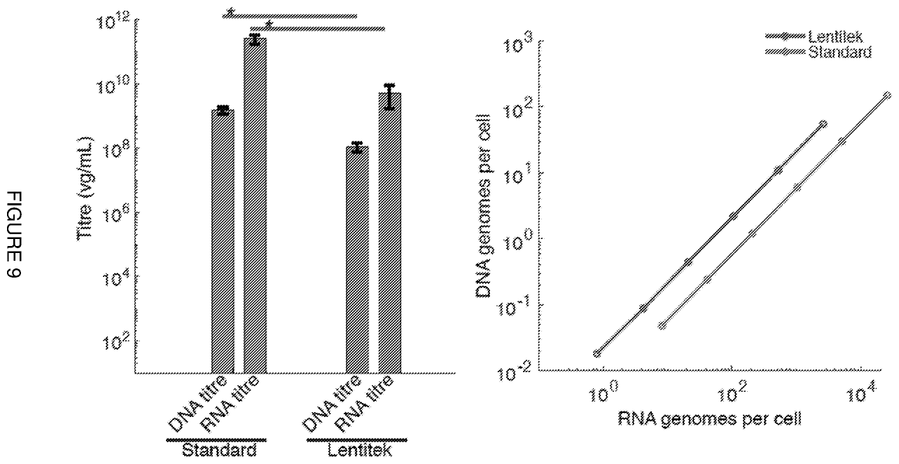

FIG. 9. Comparison of vector titres by PCR. Left panel shows qPCR viral of the viral production (RNA titre) as well as the infective titre of transformed cells (DNA titre). The right panel shows the rate of DNA provirus synthesis in the transformed cell as a proportion to RNA genome supply.

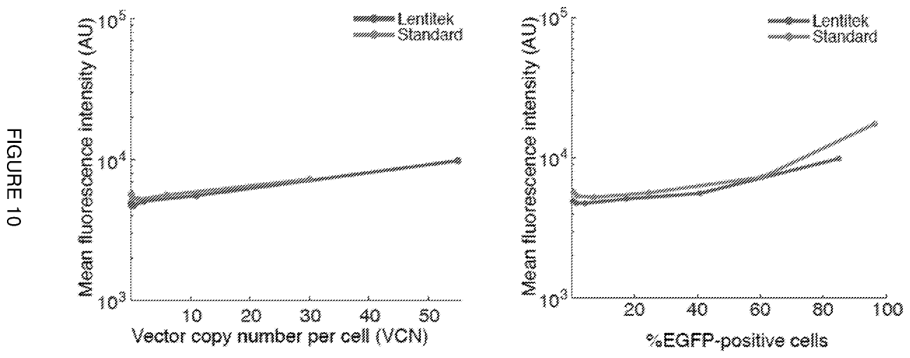

FIG. 10. Comparison of expression by flow cytometry. Left panel shows the overall fluorescence of the transformed HEK293 cells when transformed with normalised Lentitek or standard lentivirus. Right panel shows the relative fluorescence for transformed cells for either vector.

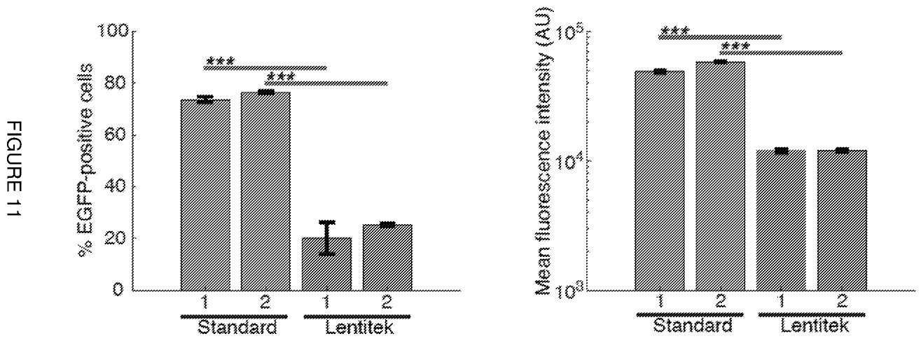

FIG. 11. Comparison of GFP expression by flow cytometry in the manufacturing cells. Left panel shows the level of cells which are expressing a detectible level of GFP. Right panel shows the levels of GFP within cells which are expressing GFP.

FIG. 12. Schematic layout of eukaryotic RNA Pol I promoter, adapted from (Pikaard, C. S., Pape, L. K., Henderson, S. L., Ryan, K., Paalman, M. H., Lopata, M. A., . . . & Sollner-Webb, B. (1990). Enhancers for RNA polymerase I in mouse ribosomal DNA. Molecular and cellular biology, 10 (9), 4816-4825.)

### MATERIALS AND METHODS

### Cloning and Molecular Biology

Standard cloning and molecular biology techniques were used to generate variants of the plasmids. These include PCR amplification from template human DNA, site directed mutagenesis, restriction enzyme digestion and topoisomerase assisted cloning. Plasmids were amplified with standard Miniprep protocols. Nucleic acid sequences were also synthesised from GeneArt (ThermoFisher).

### Lentivirus Production and Titration

VSV-G-pseudotyped lentiviral vectors were produced by co-transfecting 6×10⁶ HEK 293T cells with 2 μmol of the respective transgene plasmids along with between 0.5 and 1 μmol of the packaging and envelope plasmids. A suitable transfection agent was used to introduce the plasmids into the cell, in this case FuGENE®6 (Promega) was used as a transfection reagent at a ratio of 3 μl per 1 μg of DNA.

HEK 293T cells were incubated at standard conditions (37° C., 5% CO₂ in a humid atmosphere) in Opti-MEM® media (ThermoFisher)

Virus-containing medium was collected at 48 and 72 hours post-transfection. After each collection, the supernatant was filtered through a cellulose acetate membrane (0.45 μm pore). Lentivirus harvests were combined as required and stored at 4° C. before ultracentrifugation for 2 h at 90000×g at 4° C. Virus pellets were re-suspended in 200 μl of Opti-MEM®.

For virus titration, 1×10⁵ HEK 293T cells were plated into each well of a 6 well plate and transduced with a range of volumes of the concentrated lentivirus. Seventy-two hours after transduction, HEK 293T cell genomic DNA was extracted and the proviral titre was calculated by qPCR.

### Transduction of Cell Lines

HEK 293T cells were plated onto a flat-bottom 96 well plate at a density of 3×10⁴ cells per well. Lentiviruses were introduced 24 hours later at the appropriate MOI in a total volume of 100 μl. For dose-response analysis of NIGW, culture medium was supplemented with neomycin (Geneticin® (Life Technologies)) at a working concentration of 0.4 mg/ml.

### Flow Cytometry Detection of Transgene Expression

Cells were trypsinised and 200 μl of the suspension was added to a round bottom 96-well-plate for analysis in a BD FACSArray™ instrument. GFP fluorescence was excited with a 488 nm argon laser. During analysis of cytometry plots, live cell populations were gated by plotting forward-light-scatter versus side-scatter to visualise and isolate the viable population. GFP-positive populations were determined by plotting the emission from the green channel (detected using 530/30 nm band pass filter) against emission from the yellow channel (detected using 575/26 band pass filter), to compensate for auto-fluorescence events. Unless mentioned otherwise, non-transduced populations were used to set the baseline for GFP expression.

All FACS data were analysed by FlowJo software version 9.3.1 (©Tree Star, Inc).

### Results

### Pol I Test Plasmid

The initial test experiment used a published genome/payload plasmid with a GFP reporter as the payload: pLenti CMV GFP Puro (https://www.addgene.org/17448/). In this plasmid RSV is the Pol II promoter which drives genome transcription. In short, a comparison between Pol II and Pol I can be established by substituting the Pol II promoter upstream of the 5′ LTR and cloning in a Pol I mouse terminator immediately after the 3′ LTR sequence. The packaging plasmids were supplied as a working system.

As discussed, Pol I promoters have been utilised in the past to produce RNA transcripts for various end uses. There is however, prevailing evidence that the exact promoter sequence is species specific. Hence, for the production of Pol I driven transcripts in human cells a human Pol I promoter sequence should be utilised. There are various citations for how the Pol I promoter is cloned to produce a transcript, however little detail on the exact promoter sequence is contained within the publications or supplementary information. For example, Neumann et al use human Pol I to generate influenza transcripts, but do not detail the exact sequences used [7].

A review of sequences obtained from the databases for the human Pol I promoter sequence, seemed to confirm the correct terminal sequence to be TTATT as described by Neumann [7]. As the Genebank sequence confirmed TTATT, this sequence was chosen.

The other issue for promoter selection is the length of the sequence to include. 1 kb upstream of the TTATT motif on Genebank accession KY962518.1 was taken as the base sequence for the RNA Pol I promoter. It is highly likely that a smaller sequence is feasible, as the Sos_ZsG_pol I-mg plasmid uses a 183 base segment. Therefore, a 249 base segment was taken upstream of the TTATT motif (inclusive) as the Pol I promoter.

Unlike the promoter sequence, the RNA Pol I terminator sequence seems to be less species specific. Unlike the promoter sequence, this 78 nt sequence was readily available in the literature with no discrepancy. Where the SV40 poly (A) signal sequence is somewhat downstream of the 3′ LTR, separated by 71 bases of seeming cloning vector backbone. The Pol I terminator should immediately follow the end of the intended transcript (FIG. 3).

With these design elements in mind the pEX-K168_Lenti_CMV-GFP_Puro sequence was edited accordingly (FIG. 5).

In addition to this comparative work outlined above with GFP, the Pol I design was included into a separate Luciferase/EGFP system (FIG. 6).

### Validation of Pol I Plasmid

A small scale comparison in adherent HEK293T cells were conducted in T75 flasks. The Pol I and Pol II driven GFP payload plasmid was compared to a known control GFP payload from ViroCell. Productivity was measured through qPCR, p24 ELISA and FACS analysis.

The ELISA results in FIG. 7 show that virus is being produced to a similar level than the controls. It is well known that p24 ELISA can overestimate plasmid titre as some quantification kits can struggle to differentiate p24 which forms part of the viral particle and free p24. Therefore, whilst this result is encouraging, PCR and infectivity data from FACS are preferable.

Perhaps the most important measure of manufacturing efficiency is the infective titre. FIG. 8 shows that the Pol I promoter produces infective virus which can transform cells to express GFP.

In a parallel experiment, the Pol I promoter and terminator sequences used in the above experiment were cloned into a lab specific GFP reporter system (FIG. 6). FIG. 9 shows the PCR titre of the viral RNA and the cellular DNA post transformation.

The data in FIG. 9 shows that although less lentivirus is being produced by the Pol I method, the virus which is being produced is able to integrate into the target host DNA in a similar dose response manner to the RNA Pol II produced lentivirus. Indeed, where there appears to be a slight difference in the rate of DNA integration in FIG. 9, this is not seen in the flow cytometry analysis when adjusting for the viral concentration (FIG. 10).

Therefore, the ability for individual viruses to integrate into the target cell and subsequently express the gene of interest (GFP) is indistinguishable between Pol II produced lentivirus and Pol I produced lentivirus.

Analysis was carried out in parallel on the levels of GFP production in the manufacturing cells. The manufacturing cell line was assessed for the production of GFP prior to production of infective virus (FIG. 11). As the transgene promoter is a liver promoter, it should not express to a significant level in the HEK293 kidney cell line unless there is some breakthrough driven by upstream promoters, or some RNA splicing event. Unsurprisingly, for the CMV driven plasmid system, breakthrough is seen, where up to 80% of the cells express relatively high amounts of GFP. In contrast, the Pol I Lentitek system has a much reduced level of cells detected at 20%, and these cells are expressing approximately 6 fold lower amounts of GFP. Indeed, fluorescence in the Pol I condition was only able to be detected by the highly sensitive flow cytometry. Fluorescence microscopy was not able to capture such low levels of GFP expression in the Pol I system.

### CONCLUSION

RNA polymerase I (Pol I) is able to drive the expression of the payload plasmid to produce infective lentivirus particles. Lentivirus particles produced by the Pol I method behave in the same manner as standard methods, in that they can transform cells to the same level when accounting for viral titre. Importantly, there is a significant reduction in breakthrough expression of the payload, in this case GFP, in manufacturing cells when using the Pol I system.

### REFERENCES

1. Bulcha, J. T., Wang, Y., Ma, H., Tai, P. W., & Gao, G. (2021). Viral vector platforms within the gene therapy landscape. Signal transduction and targeted therapy, 6 (1), 1-24.
2. Perry, C., & Rayat, A. C. (2021). Lentiviral vector bioprocessing. Viruses, 13 (2), 268.
3. Johari, Y. B., Scarrott, J. M., Pohle, T. H., Liu, P., Mayer, A., Brown, A. J., & James, D. C. (2022). Engineering of the CMV promoter for controlled expression of recombinant genes in HEK293 cells. Biotechnology Journal, 2200062.
4. Osman, S., & Cramer, P. (2020). Structural biology of RNA polymerase II transcription: 20 years on. Annual Review of Cell and Developmental Biology, 36, 1-34.
5. Lizee, G., Aerts, J. L., Gonzales, M. I., Chinnasamy, N., Morgan, R. A., & Topalian, S. L. (2003). Real-time quantitative reverse transcriptase-polymerase chain reaction as a method for determining lentiviral vector titers and measuring transgene expression. Human gene therapy, 14 (6), 497-507.
6. Cordes, N., Kolbe, C., Lock, D., Holzer, T., Althoff, D., Schäfer, D., . . . & Kaiser, A. D. (2021). Anti-CD19 CARs displayed at the surface of lentiviral vector particles promote transduction of target-expressing cells. Molecular Therapy-Methods & Clinical Development, 21, 42-53.
7. Neumann, G., Zobel, A., & Hobom, G. (1994). RNA polymerase I-mediated expression of influenza viral RNA molecules. Virology, 202 (1), 477-479

1. A lentiviral genome construct comprising a lentiviral genome and a RNA polymerase I promoter, wherein expression of the lentiviral genome is under control of the RNA Polymerase I promoter.

2. The lentiviral genome construct of claim 1, wherein the construct further comprises a Pol I termination signal.

3. The lentiviral genome construct of claim 1, wherein the construct further comprises a 5′ long terminal repeat (5′ LTR) and/or a 3′ long terminal repeat (3′ LTR) and wherein the 5′ LTR and/or 3′ LTR may be self-inactivating.

4. The lentiviral genome construct of claim 1, wherein the 5′ LTR is positioned or located downstream of the RNA polymerase I (Pol I) promoter and the 3′ LTR is positioned or located upstream of the Pol I termination signal.

5. The lentiviral genome construct of claim 1, wherein the construct further comprises an insert, which insert is flanked by said 5′ and 3′ long terminal repeats.

6. The lentiviral genome construct of claim 1, further comprising a transgene.

7. A lentiviral genome construct according to claim 1 comprising:
(i) a RNA polymerase I (Pol I) promoter;
(ii) a Pol I termination signal;
(iii) a 5′ long terminal repeat (5′ LTR);
(iv) a 3′ long terminal repeat (3′ LTR); and
(v) an insert, optionally comprising a transgene, located or disposed between the 5′ and 3′ long terminal repeats;
wherein, the 5′ LTR is positioned or located downstream of the RNA polymerase I (Pol I) promoter and the 3′ LTR is positioned or located upstream of the Pol I termination signal and wherein the 5′ LTR and/or 3′ LTR may be self-inactivating.

8. A construct for use in an expression system, wherein the construct comprises:
an insert flanked by a 5′ long terminal repeat (5′ LTR) and a 3′ long terminal repeat (3′ LTR), wherein the 5′ LTR is downstream of an RNA polymerase I (Pol I) promoter and the 3′ LTR is upstream of a Pol I termination signal, wherein said insert comprises a transgene and wherein the 5′ LTR and/or 3′ LTR may be self-inactivating.

9. The construct for use of claim 8, wherein the expression system is for use in producing a recombinant retrovirus,
said insert optionally further comprising a posttranscriptional response element downstream of said transgene;
and further optionally, said insert comprising a retroviral packaging element and/or one or more nuclear export component(s) upstream of said transgene.

10. The construct for use according to claim 8, wherein the transgene of said insert comprises a nucleic acid sequence encoding one or more gene(s) of interest.

11. The construct for use according to claim 8, wherein the insert further comprises one or more additional promoter(s) for the expression of the one or more gene(s) of interest.

12. The construct for use according to claim 8, wherein the construct is inserted into a vector.

13. The construct for use according to claim 12, wherein the vector is a plasmid.

14. The construct for use according to claim 8, wherein the Pol I promoter is derived from a mammalian Pol I promoter sequence.

15. The construct for use according to claim 8, wherein the one or more additional promoter(s) for the expression of one or more gene(s) of interest comprises an RNA polymerase II promoter.

16. The construct for use according to claim 15, wherein the RNA polymerase II promoter is a CMV, RSV, SV40, EFS (EF1a short), Ubc promoter, or SFFV promoter.

17. The construct for use according to claim 8, wherein the expression system is for use in producing a recombinant lentivirus.

18. The construct for use according to claim 17, wherein the recombinant retrovirus is a non-replicating recombinant retrovirus or the recombinant lentivirus is a non-replicating recombinant lentivirus.

19. The construct according to claim 8, wherein the system-optionally further comprises one or more vector(s) comprising:
one or more viral genome packaging component(s);
one or more viral envelope protein(s);
one or more nuclear export element(s); and/or
one or more stabilising motif(s).

20. The construct of claim 19, wherein the one or more viral genome packaging component(s) comprises Gag, Pol and/or Rev.

21. The construct of claim 19, wherein the one or more viral envelope protein(s) comprises VSV-G, murine leukemia virus (MULV), the gibbon ape leukemia virus (GALV), the feline endogenous RD114 retrovirus, Moloney MULV 4070A, Moloney MULV strain 10A1, rabies virus glycoprotein, and/or the measles virus hemagglutinin and fusion glycoprotein.

22. The construct of claim 19, wherein the one or more nuclear export element(s) comprises Rev response element (RRE).

23-32. (canceled)

33. A method for producing a recombinant retrovirus, wherein the method comprises transfecting a host cell by contacting the said host cell with the construct of claim 1, and one or more vector(s) encoding one or more additional viral components; culturing the host cell under conditions in which the recombinant retroviral vector particles are produced; and isolating said recombinant retroviral vector particles.

34. The method of claim 33, wherein the recombinant retrovirus is a recombinant lentivirus.

35. (canceled)

36. The method of claim 33, wherein the one or more additional viral components are selected from: (i) one or more packaging component(s); and/or (ii) one or more envelope protein(s);

37. The method of claim 33, wherein the one or more packaging component(s) comprises Gag, Pol and/or Rev, and the envelope protein comprises VSV-G, murine leukemia virus (MULV), the gibbon ape leukemia virus (GALV), the feline endogenous RD114 retrovirus, Moloney MULV 4070A, Moloney MULV strain 10A1, rabies virus glycoprotein, and/or the measles virus hemagglutinin and fusion glycoprotein.

38. (canceled)

39. The method of claim 33, wherein the method further comprising selecting cell clones in which the various lentiviral genetic elements have stably integrated into the cell's genome.

40. (canceled)


SEQUENCE LISTING (8 sequences)

SEQ ID NO: 1 - DNA, 38 bp, synthetic construct
  source 1..38: mol_type = other DNA
tttcgctccgagtcggcattttgggccgccgggttatt

SEQ ID NO: 2 - DNA, 250 bp, synthetic construct
  source 1..250: mol_type = other DNA
ggccggccccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccgggggaggtatatctttcgctccgagtcggcattttgggccgccgggttatt

SEQ ID NO: 3 - DNA, 78 bp, synthetic construct
  source 1..78: mol_type = other DNA
tcccccccaacttcggaggtcgaccagtactccgggcgacactttgttttttttttttcccccgatgctggaggtcga

SEQ ID NO: 4 - DNA, 18 bp, synthetic construct
  source 1..18: mol_type = other DNA
gctgacacgctgtcctct

SEQ ID NO: 5 - DNA, 1000 bp, synthetic construct
  source 1..1000: mol_type = other DNA
gaggggctgcgttttcggcctcgggaagagcttctcgactcacggtttcgctttcgcggtccacgggccgccctgccagccggatctgtctcgctgacgtccgcggcggttgtcgggctccatctggcggccgctttgagatcgtgctctcggcttccggagctgcggtggcagctgccgagggaggggaccgtccccgctgtgagctaggcagagctccggaaagcccgcggtcgtcagcccggctggcccggtggcgccagagctgtggcgcgtcgcttgtgagtcacagctctggcgtgcaggtttatgtgggggagaggctgtcgctgcgcttctgggcccgcggcgggcgtggggctgcccgggccggtcgaccagcgcgccgtagctcccgaggcccgagccgcgacccgcggggacccgccgcgcgtggcgcgggaggctggggacgcccttcccggcccggtcgcgggtccgcgctcatcctggccgtctgaggcggcggccgaattcgtttccgagtccccgtggggagccggggaccgtcccgcccccgtcccccgggtgccggggagcggtccctctgccgcgatcctttctggcgagtccccgtgcggagtcggagagcgctccctgagcgcgcgtgcggcccgagaggtcgcgcctggccggccttcggtccctcgtgtgtcccggtcgtaggaggggccggccgaaaatgcttccggctcccgctctggagacacgggccggccccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccgggggaggtatatctttcgctccgagtcggcattttgggccgccgggttatt

SEQ ID NO: 6 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 7 - DNA, 234 bp, synthetic construct
  source 1..234: mol_type = other DNA
tggaagggctagctcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 8 - DNA, 351 bp, synthetic construct
  source 1..351: mol_type = other DNA
atggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag